(19) United States
(12) Patent Application Publication
(10) Pub. No.: US 2024/0181124 A1
(43) Pub. Date: Jun. 6, 2024

(54) COMPOSITIONS COMPRISING POROUS MICROPARTICLES AND METHODS OF USING THE SAME

(71) Applicant: WEST CHESTER UNIVERSITY, West Chester, PA (US)

(72) Inventor: Kurt W. Kolasinski, West Chester, PA (US)

(73) Assignee: WEST CHESTER UNIVERSITY, West Chester, PA (US)

(21) Appl. No.: 18/525,295

(22) Filed: Nov. 30, 2023

Related U.S. Application Data
(60) Provisional application No. 63/429,520, filed on Dec. 1, 2022.

(51) Int. Cl. A61L 26/00

(52) U.S. Cl. CPC A61L 26/0004; A61L 26/0066; A61L 2300/104; A61L 2300/406

(57) ABSTRACT

The present invention relates to compositions comprising microparticles and methods of using the same. Provided are compositions comprising a nanostructured material with tortuous pores. Also provided are compositions comprising a nanostructured material comprising metal. Pharmaceutical compositions comprising the compositions disclosed herein are also disclosed. The compositions and pharmaceutical compositions disclosed herein enhance wound healing properties of solutions, dispersions, creams, gels, or dressings applied to wounds. Also provided are methods of using the disclosed compositions and pharmaceutical compositions.

### STATEMENT REGARDING FEDERALLY SPONSORED RESEARCH

This invention was made with government support under Grant No. CMMI-182533 awarded by the National Science Foundation. The government has certain rights in the invention.

### FIELD OF THE INVENTION

The present invention relates generally to a nanostructured porous material with customizable surface chemistry and methods for the treatment or prevention of bacterial infections in a mammal. The invention also includes methods of treating bacterial infections in a mammal by administering to an infected area of the mammal a pharmaceutical composition of the present invention.

### BACKGROUND

Wound healing is a complex process involving wound closure, generation of new tissue with sufficient vascularization and suppression of infection. There are no clean wounds. All wounds contain some degree of bacterial contamination. A healthy immune system can usually deal with this microbial intrusion; however, if the wounds are extensive or in immunocompromised, elderly, or chronically ill patients, e.g., diabetics, additional assistance is required to overcome an infection. Biofilm formation is one of the primary causes of chronic bacterial and implant-associated infections. Wounds also comprise a broad range of characteristics requiring and equally broad range of therapeutic approaches. Treatment recommendations highlight the importance of topical antiseptic treatment and custom dressings. While a wide range of commercial chemical dressings are on the market, there is a no efficient intervention in order to deal with the dysfunctional wound healing processes.

One approach to combat infections utilizes nitric oxide (NO). NO can trigger the dispersal of biofilms as well as the immune system response to infections (Sadrearhami et al., 2018). As such NO exhibits both killing and dispersal activities against microbial biofilms. A biological lifetime on the order of seconds limits the action of NO to <0.5 mm from the point of generation/release; therefore, effective dosage requires application of a delivery vehicle. Currently, there is no delivery vehicle for NO. Tissue adhesion is responsive to both the structure and surface chemistry of particles that contact the tissue. Endothelial cell adhesion and proliferation are enhanced by surface roughness (Zawislak et al., 2009). The rough texture of porous Si enhances the proliferation of endothelial cells (Dalilottojari et al., 2016). Sustained release of growth factors has been achieved by loading into a porous Si scaffold that has been epoxy functionalized by plasma polymer coating. Vera Vec endothelial cell proliferation was enhanced compared to flat microarray substrates (Dalilottojari et al., 2016). Porous Si particles were loaded with ciprofloxacin (CIP). Exposure of the CIP-loaded por-Si to a wound with elevated pH caused the por-Si to oxidize and resulted in instant release of CIP (Chen et al., 2017).

Diabetes is a major cause of chronic wounds and as many as one quarter of diabetics will suffer from diabetic foot ulcers. These ulcers are a leading cause of hospitalization and a major source of morbidity among diabetics. Current wound care recommendations rely on topical antiseptic treatments and custom dressings. Antiseptic treatments must balance the cytotoxicity of antiseptics such as povidone-iodine, chlorhexidine, hydrogen peroxide, boric acid, silver sulfadiazine or nitrate, sodium hypochlorite, and mafenide acetate with the potential for deleterious host cell interactions that can negatively affect wound healing. (Jahromi et al., 2018)

Conventional delivery systems such as gels release an active pharmaceutical ingredient (API) over a period no longer than 1-2 days. For example, Regen-D 150 (Bharat Biotech) is a gel containing recombinant human epidermal growth factor that requires application twice daily for 15 to 20 weeks until the diabetic foot ulcer is healed.

Nanostructured materials that interact with API to enhance delivery are known as nanocarriers. Nanocarriers have the potential to improve outcomes by enhancing the closing of wounds, suppressing infection, and stabilizing API. Examples of the use of nanotechnology to address wound care can be found in the prior art; however, translation of nanomedicine research into effective therapies remain challenging (Ashtikar and Wacker, 2018).

ACTICOAT™ (Smith+Nephew) is a silver nanoparticle containing dressing indicated for burns and chronic wound care (Burrell et al., 1999). It releases silver ions to provide antibacterial activity over a period of up to seven days (Ashtikar and Wacker, 2018). Polymem® silver (Ferris Mfg. Corp.) is another nanocrystalline silver dressing. While silver nanoparticle dressings are mainly indicated for treatment of burns, chronic and surgical wounds, they are expensive with total average treatment cost reported to exceed €13,000 (Ashtikar and Wacker, 2018). In addition, there are concerns regarding the risks associated with long-term exposure and accumulation of nanoparticles, such as silver, that are non-resorbable and for which there is no dietary need.

The shelf life of therapeutic formulations is an important characteristic as short shelf-life is not only inconvenient but also it translates into greater cost and reduced availability. This is of acute importance in markets throughout the world in which, for example, access to refrigeration for on demand delivery are limited.

### SUMMARY OF EMBODIMENTS

The disclosure relates to compositions comprising a nanostructured material with tortuous pores and, in some embodiments, a nanostructured material comprising metal. The composition enhances wound healing properties of solutions, dispersions, creams, gels, or dressings applied to wounds including, for example, ulcers, abrasions, punctures, and lacerations. The nanostructured material is resorbable with self-replicating surface roughness that aids in wound closure by promoting tissue adhesion and cell proliferation. The nanostructured material exhibits antibacterial action that can be enhanced by the addition of immobilized metal nanoparticles. The dissolution products of this resorbable material promote angiogenesis and the formation of healthy tissue during wound healing. In addition, the surface chemistry of this material is controllable, for example, producing a surface with either positive or negative zeta potential to facilitate the optimization of cell-surface interactions to promote wound healing. Control of the surface chemistry of pore walls facilitates stabilization of active pharmaceutical ingredients (API) added to these formulations, which can enhance shelf life of formulations. Encapsulation within pores and physisorption onto surfaces with appropriate surface chemistry are potential pathways for carriers to stabilize and enhance the shelf life of API.

The disclosure relates to compositions comprising a solid substrate comprising silicon greater than about 90% in weight relative to total weight of the solid substrate and greater than about 0.1% weight in oxygen relative to the total weight of the solid substrate; wherein the solid substrate is in the form of a microparticle comprising an exterior surface and one or a plurality of pores defining one or a plurality of interior surfaces; wherein the solid substrate is no more than about 500 microns at its greatest length; and wherein at least about 50% of the exterior surface comprises protrusions and/or a relative roughness of about 1% to about 10%.

In some embodiments, the exterior surface comprises a metal. In some embodiments, the metal is adhered onto the exterior surface in the form of nanoparticles. In some embodiments, the metal is copper, silver, gold, platinum or palladium.

In some embodiments, the solid substrate comprises silicon at about 98% in weight relative to total weight of the solid substrate. In some embodiments, the solid substrate comprises silicon at about 99% in weight relative to total weight of the solid substrate and wherein the exterior surface comprises nanoparticles of metal. In some embodiments, the microparticle is a silicon microparticle of oblongate or amorphous shape that has pores or cavities on its outer surface and asperities. In some embodiments, the pores and exterior surface is a torutuous surface with a plurality of asperities that range in height and depth.

In some embodiments, the exterior surface comprises a region of hydrophilicity. In some embodiments, the solid substrate in an aqueous solution has a zeta potential from about +20 to about +80 mV. In some embodiments, the region of hydrophilicity across the exterior surface comprises polar adsorbates. In some embodiments, the positively charged ions are chosen from arginine, amino acids, carboxylic acids, alcohols, thiols, dyes, a molecule comprising a carboxylic acid group, or salts thereof. In some embodiments, the solid substrate is in powdered form. In some embodiments, the exterior surface comprises a net positive charge. In some embodiments, the exterior surface comprises is modified with covalently bound alcohol, carboxyilic acids, thiols or positively charge amino acids, such as arginine. In some embodiment the exterior surface comprises a dye that intercalates with the porous silicon or silicon dioxide that is the solid substrate of the exterior surface.

In some embodiments, the microparticle comprises a pore volume of from about 0.05 to about 1.9 cm³ per gram of mass of the microparticle. In some embodiments, the microparticle Brunauer, Emmett, Teller (BET) comprises a surface area of from about 20 to about 900 centimeters squared per gram. In some embodiments, the microparticle is amorphous in shape but pseudo-rectangular in shape (plate-like and tending toward rectangular) and comprises a longest dimension in length from about 1 to about 75 microns. In some embodiments, the microparticle has a longest dimension across of about 7.5 microns. In some embodiments, the ratio of height to length at longest dimension of the microparticle is from about 1:4 to about 1:20. In some embodiments, the microparticle comprises a plurality of asperties in a tortuous pattern with a height of one or a plurality of an asperity comprising more than about 5% of the longest length.

In some embodiments, the microparticle comprises copper and a weight-to-weight ratio copper to silicon of from about 0.1 to about 7.0%. In some embodiments, copper ions are sprayed, deposited or adhered to the exterior surface of the microparticle. In some embodiments, copper is sprayed, deposited or adhered onto the exterior surface of the microparticle such that the metal ion adheres to the surface and evenly dispersed or substantially evenly dispersed throughout the surface area of the exterior surface of the microparticle. In some embodiments, the exterior surface of the microparticle is covalently or non-covalently bound to a therapeutic agent.

The disclosure also relates to pharmaceutical compositions comprising: (i) the composition comprising silicon microparticles disclosed herein; and (ii) a pharmaceutically acceptable carrier. In some embodiments, the composition is in a solid dosage form or a liquid dosage form; and wherein the solid substrate is at a weight from about 0.1% to about 5.0% weight-to-weight ratio of the solid substrate to total weight of the pharmaceutical composition.

In another aspect, provided herein are methods of treating a bacterial infection in a subject in need thereof comprising administering to the subject a therapeutically effective amount of the pharmaceutical compositions disclosed herein. In some embodiments, the step of administering is administering intravenously, topically, irrigation of wounds either as part of a wound dressing or in sterile solution, intradermally, intramucosally, subcutaneously, sublingually, orally, intravaginally, intramuscularly, intracavernously, intraocularly, intranasally, into a sinus, intrarectally, gastrointestinally, intraductally, intrathecally, subdurally, extradurally, intraventricular, intrapulmonary, into an abscess, intra articular, into a bursa, subpericardially, intrauterine, into the pleural space, transmucosally, or transdermally.

In another aspect, provided herein are methods of enhancing or accelerating wound healing in a subject in need thereof comprising exposing pharmaceutical compositions of the present disclosure to a wound in the subject.

In another aspect, provided herein are methods of treating dermatitis in a subject in need thereof comprising administering to the subject a therapeutically effective amount of the pharmaceutical compositions of the present disclosure to the subject at the site of dermatitis. In some embodiments, the dermatitis is topical dermatitis.

In another aspect, the disclosure relates to methods of making the compositions of the present disclosure comprising etching a composition of silicon with an initial amount of a first oxidant reactive with silicon. In some embodiments, the initial amount of the first oxidant is from about 0.1% to about 5% of a stoichiometric amount required for etching. In some embodiments, a second oxidant is injected over time and wherein the concentration of the second oxidant does not exceed 50%. In some embodiments, the first oxidant is an oxovanadium (V) generating species or a soluble metal salt and the second oxidant is hydrogen peroxide. In some embodiments, the oxovanadium (V) generating species is V₂O₅. In some embodiments, the soluble metal salt is copper(II) nitrate, copper(II) sulfate, silver(I) nitrate, silver(I) acetate, palladium(II) chloride, chloroauric acid or hexachloroplatinic acid. In some embodiments, the method is free of anodic etching. In some embodiments, the method comprises injection Metal-Assisted Chemical Etching (iMACE) comprising injecting hydrogen peroxide.

### BRIEF DESCRIPTION OF THE DRAWINGS

FIG. 1: Bright field (BF) micrographs obtained by transmission electron microscopy (TEM) of 3-4 nm tortuous pores in ReEtched porous silicon.

FIGS. 2A and 2B: High-angular annular dark field (HAADF) micrographs obtained by transmission electron microscopy (TEM). The copper nanoparticles used to catalyze low-load metal assisted catalytic etching (LL-MACE) are clearly visible in image (A). The tortuous nature of both the etch track pores in image (A) and the surrounding mesopores in image (B) is exhibited. This contrasts with the parallel linear etch track pores observed in high-load metal assisted catalytic etching (HL-MACE).

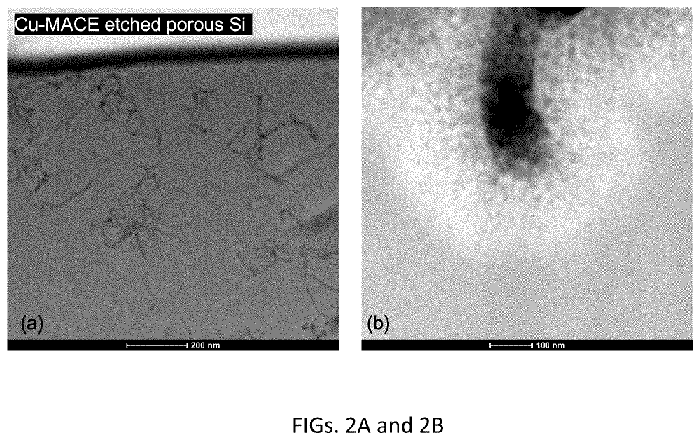

FIGS. 3A-3C: Secondary electron micrographs of silicon particles obtained by scanning electron microscopy (SEM). (A) An image of bulk silicon particles after grinding exhibits plate like particles with generally flat surfaces caused by cleavage of polycrystalline particles along crystallographically defined directions. (B) An image of the rough surface of silicon particles after porosification with LL-MACE. (C) An image of porous silicon particles ground into nanoparticles that exhibit rough surfaces caused by fracturing along tortuous pores.

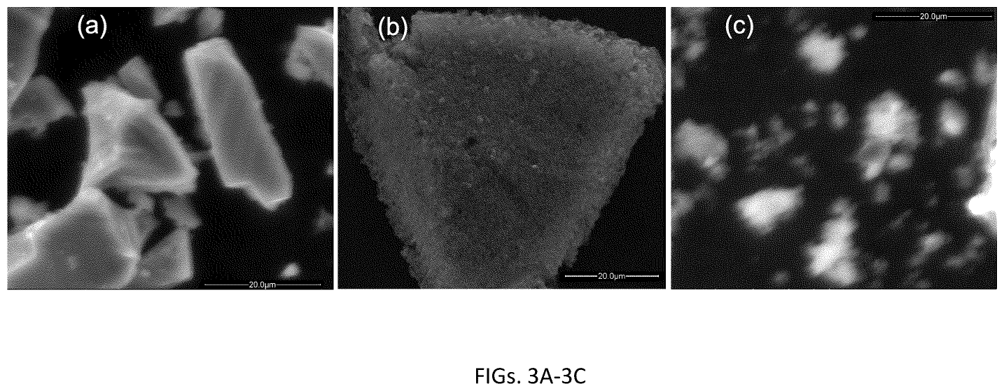

FIG. 4: Infrared (IR) absorption spectrum of porous silicon that has been modified by mechanochemistry to terminate external surfaces with arginine fragments, as evinced by the formation of Si—O bonds and the signature of NH₂ moieties in the absence of OH moieties. Si—H groups, which were initially formed by etching on internal pore surfaces, are maintained after mechanochemical transformation.

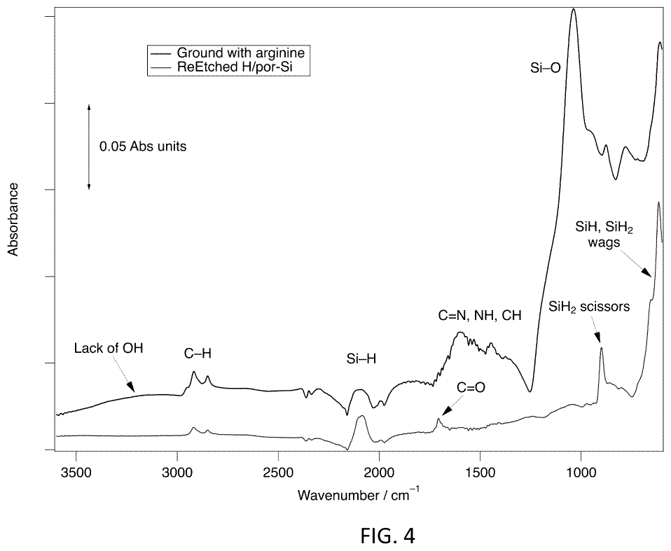

FIG. 5: Concentration profile for the release of ibuprofen from Cu-MACE etched porous silicon into phosphate buffered saline solution (PBS) at room temperature. The profile is consistent with burst release of ibuprofen from the exterior surface of the porous silicon over the first 24 h followed by much slower diffusive release from the interior pore volume.

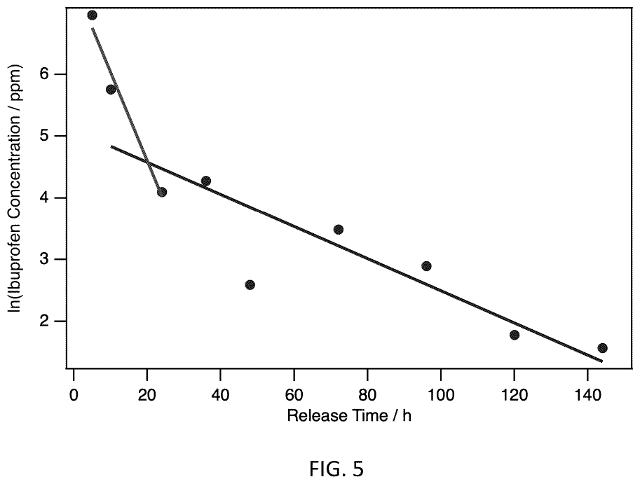

FIG. 6 illustrates the injection MACE cycle for etching silicon (Si), denoted iMACE. Injected H₂O₂ removes an electron from a metal nanoparticle, M, which then injects a hole into the silicon substrate. The hole initiates the etching of silicon forming the etch product SiF₆²⁻, to form a pore in the substrate.

### DETAILED DESCRIPTION OF EMBODIMENTS

Some of the main embodiments of the present disclosure are described in the above Summary section of this application, as well as in the Examples, Figures, and Claims. This Detailed Description of Embodiments section provides additional description relating to the compositions and methods of the present disclosure and is intended to be read in conjunction with all other sections of the present patent application, including the Summary, Examples, Figures, and Claims sections of the present application.

### I. Definitions

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary, Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one 01” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. As used in the specification and in the claims, the term “comprising” can include the aspects “consisting of” and “consisting essentially of.” Comprising can also mean “including but not limited to.”

As used herein, the term “microparticle” means a particle of matter from about 0.5 nanometers and about 500 microns in dimension at its longest dimension. If the particles are spherical or roughly spherical, a microparticle may be defined by its longest dimension of diameter across the longest longitudinal plane across the particle. Microparticles are available in a wide variety of materials, including ceramics, glass, polymers, and metals. In some embodiments, microparticles of the disclosure comprise a solid substrate that defines its shape. In some embodiments, the solid substrate is solid silicon. In some embodiments, the solid substrate comprises silicon, silicon suboxide SiOₓ in which x<2, silicon dioxide or combinations thereof. In some embodiments, the microparticle is in a wafer form or is in a powdered form crushed or broken from a solid wafer form after being produced. In some embodiments, the solid substrate is sprayed onto or exposed to metal ions, such as copper, such that the metal ions, such as copper, adhere to at least one region of the exterior surface of the microparticle. In some embodiments, the microparticle comprises a rough exterior surface defined by a plurality of asperities across more than about 50%, 60%, 70%, 80%, 90%, 91%, 92%, 93%, 94%, 95%, 96%, 97%, 98%, 99% or 100% of its exterior surface. In some embodiments, the microparticle comprises one or a plurality of pores or cavities. The pores or cavities define a contiguous surface within which one or a plurality of asperities may, optionally, also exist. The interior surface of the microparticle ends at the interface between an edge of a pore or cavity and the rest of the surface of the particle that mostly faces to a point away from the particle surface. In some embodiments, the interior surface of the microparticle ends at the interface between an edge of a pore or cavity and a point on the surface of the microparticle facing on a vector opposite to a vector pointing toward interior of the particle. In some embodiments, the interior surface of the microparticle comprises one or a plurality of asperities, such asperities increasing the roughness of the microparticle surface.

As used herein, the term “nanoparticle” means a microparticle with an elongate, amorphous, spherical or pseudo-spherical shape (e.g. tending toward spherical) that, at its longest dimension across a longitudinal plane is no more than about 999 nanometers in length. In some embodiments, the disclosure relates to a nanoparticle or a plurality of nanoparticles, whose longest dimension across a longitudinal plane is no more than about 999, 900, 800, 700, 600, 500, 450, 400, 350, 300, 250, 200, 150, 100 nm in length. In some embodiments, the disclosure relates to a nanoparticle or a plurality of nanoparticles, whose longest dimension across a longitudinal plane is from about 1 to about 100 nm in length. In some embodiments, the composition of the disclosure comprises a heterogeneous population of microparticles, wherein the population comprises one or a plurality of silicon nanoparticles comprising an exterior surface, wherein the nanoparticles comprise one or a plurality of pores, each of the one or plurality pores defined by an interior surface, wherein the interior surface is contiguous about the volume of the pore, and wherein the exterior surface and/or interior surface comprise a metal, such as copper, palladium, platinum, silver or gold. In some embodiments, the composition comprises one or a plurality of microparticles or nanoparticles of the disclosure in a powdered form. In some embodiments, the composition comprises one or a plurality of microparticles and nanoparticles of the disclosure in a powdered form.

As used herein, the term “pore” means a space or cavity in a solid substance of any kind that contributes to porosity of the substance.

As used herein, the term “zeta potential” means the electrical potential at the slipping plane. This plane is the interface that separates mobile fluid from fluid that remains attached to the surface of a substance. A Zeta Potential Analyzer can measure the zeta potential of a solid, fibers, or powdered material. Zeta potential can also be calculated using theoretical models, and an experimentally determined electrophoretic mobility or dynamic electrophoretic mobility. Electrophoresis is used for estimating zeta potential of particulates, whereas streaming potential/current is used for porous bodies and flat surfaces. In practice, the zeta potential of dispersion is measured by applying an electric field across the dispersion. Particles within the dispersion with a zeta potential will migrate toward the electrode of opposite charge with a velocity proportional to the magnitude of the zeta potential. This velocity is measured using the technique of the laser Doppler anemometer. The frequency shift or phase shift of an incident laser beam caused by these moving particles is measured as the particle mobility, and this mobility is converted to the zeta potential by inputting the dispersant viscosity and dielectric permittivity, and the application of the Smoluchowski theories. In some embodiments, microparticles of the disclosure comprise a zeta potential of from about +20 mV to about +80 mV.

As used herein “relative roughness” is calculated by dividing the roughness size by the size of the particle. For example, a 100 μm particle having a relative roughness of from about 1% and about 10% has a roughness from about 1 μm to about 10 μm and a 300 nm particle having a relative roughness of from about 1% and about 10% has a roughness of from about 3 nm and about 30 nm.

As used herein, the phrase “in need thereof” means that the subject has been identified as having a need for the particular method or treatment. In some embodiments, the identification can be by any means of diagnosis. In any of the methods and treatments described herein, the subject is an animal or mammal in need of a treatment. In some embodiments, the animal or mammal is in an environment or will be traveling to an environment in which a particular disease, disorder, or condition is prevalent.

As used herein, the terms “treat,” “treating,” and “treatment” encompass a variety of activities aiming at desirable changes in clinical outcomes. For example, the term “treat”, as used herein, encompasses any detectable improvement in one or more clinical indicators of a bacterial infection or improvement in the rate or state of wound healing, including treating bacterial infections associated with wounds. For example, such terms encompass alleviating, abating, ameliorating, relieving, reducing, inhibiting or slowing at least one clinical indicator or symptom, preventing additional clinical indicators or symptoms, reducing or slowing the progression of one or more clinical indicators or symptoms, causing regression of one or more clinical indicators or symptoms, relieving a condition caused by the disease or disorder, and the like. In the case of therapeutic treatments, the methods and compositions provided herein can be used in subjects that already exhibit one or more wounds, that is infected with or is susceptible to infection with bacteria such as pathogenic gram negative or gram-positive bacterial strains. In the case of wounds and/or bacterial infections, various clinical indicators and symptoms are known to medical practitioners and those of skill in the art, such as the state or rate of rate of swelling, edema, the presence of absence of white blood cells, the elevated presence of lymphocytes in blood of a subject, fever, or the like. In some embodiments, the clinical indicator of a therapeutic being effective is the shrinkage of the size of an abscess, wound, or other infected area of the subject being administered one or more of the pharmaceutical compositions disclosed herein.

The terms “prevent” or “preventing” as used herein encompasses stopping a disease, disorder, or symptom from starting, as well as reducing or slowing the progression or worsening of a disease or disorder. For example, “preventing” bacterial infection includes, but is not limited to, inhibiting the progression of wound severity and/or bacterial growth within a wound or on the skin of the subject. In the case of prophylactic treatments, the methods and compositions provided herein can be used to prevent progression or clinical exhibiting of symptoms in subjects that do not yet exhibit any clear or detectable clinical indicators or symptoms of the disease or disorder but that are believed to be at risk of developing the disease or disorder, such as a wound and/or bacterial infection.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term subject. The term “subject” is also used throughout the specification in some embodiments to describe an animal from which a cell sample is taken or an animal to which a disclosed composition or pharmaceutical composition has been administered. In some embodiment, the animal is a human. For treatment of those conditions that are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study. In some embodiments, as used herein, the term “therapeutically effective amount” is meant to refer to an amount of an active agent (such as the disclosed composition or pharmaceutical composition) or combination of agents effective to ameliorate, delay, or prevent the symptoms, prevent progression of a wound and/or bacterial infection, or prolong the survival of the patient being treated for a wound and/or a bacterial infection. Determination of a therapeutically effective amount is well within the capabilities of those skilled in the art, especially in light of the detailed disclosure provided herein. In some embodiments, an effective amount may be that amount of substance shown to reduce growth or decontaminate a population of pathogenic bacteria on a surface. This may be demonstrated by reduction in colony number on agar plates seeded with a sample, such as a swipe, taken from such a surface or demonstration of no growth at all when a sample is brushed, touched or rubbed onto an agar plate with medium conducive to growing bacteria. In some embodiments, an effective amount of one or more pharmaceutical compositions of the present disclosure, sufficient for achieving a therapeutic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day.

In some embodiments, the pharmaceutical compositions of the present disclosure can be co-administered with other agents or therapeutics, such as anti-bacterial or anti-inflammatory agents. In some embodiments, the antibacterial agent is a soap, detergent or other hydrophobic substance that destroys or promotes destruction of bacterial walls. In some embodiments, the anti-inflammatory agent is a non-steroidal anti-inflammatory agent (such as ibuprofen, acetaminophen) or a steroid. In some embodiments, the pharmaceutical composition comprises a microparticle or nanoparticle that is free of any small molecule or drug covalently bound to its surface. In some embodiments, the exterior surface consists of silicon, oxidized forms of silicon and one or a plurality of metal elements, such as copper.

The disclosure relates to methods of treatment of bacterial infection in a subject in need thereof or methods of treating dermatitis in the subjects disclosed herein comprising administering to the subject a therapeutically effective amount of a pharmaceutical composition disclosed herein and a therapeutic agent. In some embodiments, the co-administration of therapeutics can be sequential in either order or simultaneous. In some embodiments, pharmaceutical compositions are co-administered with more than one additional therapeutic. Examples of therapeutics include an antibiotic (such as amoxicillin, penicillin, bactrim, clindamycin or derivatives thereof), opioid (such as lidocaine and derivatives thereof), anti-inflammatory agent (such as ibuprofen) or wound healing agent (such as vitamin E).

When used in as part of the combination therapy the therapeutically effective amount of the inhibitor may be adjusted such that the amount is less than the dosage required to be effective if used without other therapeutic procedures.

In some embodiments, administration of the pharmaceutical compositions described herein is preceded by surgical intervention.

The term “pharmaceutical composition” as used herein refers to a composition comprising at least one active agent as described herein (such as, for example, a silicon microparticle, and one or more other components suitable for use in pharmaceutical delivery such as carriers, stabilizers, diluents, dispersing agents, suspending agents, thickening agents, excipients, and the like. The disclosure relates to a pharmaceutical composition comprising an effective amount of any particle or porous microparticle and a pharmaceutically acceptable carrier. In some embodiments, the pharmaceutical composition comprises a plurality of microparticles wherein at least one microparticle is free of one or a plurality of smooth exterior surfaces.

The term “active agent” as used herein refers to a molecule that is intended to be used in the compositions and methods described herein and that is intended to be biologically active, for example for the purpose of treating wounds and/or bacterial infections. The term “active agent” is intended to include molecules that either are, or can be converted to a form that is, biologically active. For example, the term “active agent” includes pro-drugs and/or molecules that are inactive or lack the intended biological activity but that can be converted to a form that is active or has the intended biological activity.

As used herein, the term “sample” refers generally to a limited quantity of something that is intended to be similar to and represent a larger amount of that thing. In the present disclosure, a sample is a collection, swab, brushing, scraping, biopsy, removed tissue, or surgical resection that is to be tested for clinical indicators of a disease or disorder. In some embodiments, samples are taken from a surface to test whether the surface is contaminated with one or more strain of pathogenic gram negative or gram-positive bacteria. In some embodiments, the sample is taken from a patient or subject that is believed to have a bacterial infection. In some embodiments, a sample believed to contain clinical indicators of a disease or disorder, such as one or a plurality of pathogenic bacterial cells, is compared to a control sample that is known to be free of one or a plurality of clinical indicators of a disease or disorder, such as a population of pathogenic bacterial cells that would be at a concentration or number that would be sufficient to infect a subject. In some embodiments, a sample believed to contain a clinical indicator of a disease or disorder is compared to a control sample that is known to not contain a clinical indicator of a disease or disorder. In some embodiments, a sample believed to contain a clinical indicator of a disease or disorder, such as is compared to a control sample that contains the same known amount of clinical indicators of a disease or disorder.

The term “scavenge” as used herein means uptake or chemically combine with and transport to including, but not limited to, the kidney for excretion.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts of the embodiments include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising one or may be formed by reacting the free base, or a salt, enantiomer or racemate thereof, with one or more equivalents of the appropriate acid. In some embodiments, pharmaceutical acceptable salts of the present disclosure refer to amino acids having at least one basic group or at least one basic radical. In some embodiments, pharmaceutical acceptable salts of the present disclosure comprise a free amino group, a free guanidino group, a pyrazinyl radical, or a pyridyl radical that forms acid addition salts. In some embodiments, the pharmaceutical acceptable salts of the present disclosure refer to amino acids that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromaticaliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water).

The term “soluble” or “water soluble” refers to solubility that is higher than 1/100,000 (mg/ml). The solubility of a substance, or solute, is the maximum mass of that substance that can be dissolved completely in a specified mass of the solvent, such as water. “Practically insoluble” or “insoluble,” on the other hand, refers to an aqueous solubility that is 1/10,000 (mg/ml) or less. Water soluble or soluble substances include, for example, polyethylene glycol. In some embodiments, the polypeptide described herein may be bound by polyethylene glycol to better solubilize the composition comprising the peptide.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be at least about 10, 20, 30, 40, 50, 60, 70, 80, 90, 100%, or any amount of reduction in between as compared to native or control levels. In an aspect, the inhibition or reduction is about 10 to about 20, from about 20 to about 30, from about 30 to about 40, from about 40 to about 50, from about 50 to about 60, from about 60 to about 70, from about 70 to about 80, from about 80 to about 90, or from about 90 to about 100% in inhibition or reduction as compared to native or control levels. In the case of some embodiments, the control would be the amount of reduction or inhibition measured without administration of one or a plurality of pharmaceutical compositions disclosed herein. In some embodiments, the inhibition or reduction is about 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “determining” can refer to measuring or ascertaining a quantity or an amount or a change in activity. For example, determining the amount of a disclosed nucleotide in a sample as used herein can refer to the steps that the skilled person would take to measure or ascertain some quantifiable value of the nucleotide in the sample. One of ordinary skill in the art is familiar with the ways to measure an amount of the disclosed nucleotides or bacterial cells in a sample. In some cases a number or presence of pathogenic bacterial strains may be determined by detecting the presence, absence or quantity of bacterial cells in a sample by performing quantitative or semi quantitative polymerase chain reaction (PCR) and the number of copies of nucleic acid sequences found in a corresponding to a pathogenic bacterial cell would be indicative of the presence of a bacterial cell infection as compared to a control sample whose PCR would not yield the same number or presence of such nucleic acid sequences.

Additional definitions and abbreviations are provided elsewhere in this patent specification or are well known in the art.

### Compositions

The compositions and pharmaceutical compositions of the present disclosure relate to porous microparticles and, in some embodiments, porous silicon (por-Si) microparticles, and methods of using the microparticles for treatment of skin or wound of a subject, decontamination of surfaces, and treatment of dermatitis in a subject. In some embodiments, the microparticles comprise no less than about 90% silicon in weight ratio as compared to the total weight of the microparticle. In some embodiments, the microparticles comprises one or a plurality of pores or cavities dispersed throughout the exterior surface of the microparticle. In some embodiments, the exterior surface of the microparticle is tortuous or rough in texture and comprises no less than about 50% of its surface are covered in asperities or amorphous protrusions. In some embodiments, the compositions or pharmaceutical compositions comprise porous silicon microparticles disclosed herein and copper microparticles.

Microparticles of the disclosure have, in some embodiments, amorphous shape and heterogeneous in size and shape. In some embodiments, the longest dimension across the microparticle is about 0.1 micron. In some embodiments, the longest dimension across the microparticle is about 0.5 microns. In some embodiments, the longest dimension across the microparticle is about 1 micron. In some embodiments, the longest dimension across the microparticle is about 2.5 micron. In some embodiments, the longest dimension across the microparticle is about 5 microns. In some embodiments, the longest dimension across the microparticle is about 7.5 microns. In some embodiments, the longest dimension across the microparticle is about 10 microns. In some embodiments, the longest dimension across the microparticle is about 12.5 microns.

In some embodiments, the longest dimension across the microparticle is from about 0.1 microns to about 0.5 microns. In some embodiments, the longest dimension across the microparticle is from about 0.5 microns to about 1 micron. In some embodiments, the longest dimension across the microparticle is from about 1 micron and to about 2.5 microns. In some embodiments, the longest dimension across the microparticle is from about 2.5 microns to about 5 microns. In some embodiments, the longest dimension across the microparticle is from about 5 microns to about 7.5 microns. In some embodiments, the longest dimension across the microparticle is from about 7.5 microns to about 10 microns. In some embodiments, the longest dimension across the microparticle is from about 10 microns to about 12.5 microns.

In some embodiments, the ratio of height to length across a longest dimension of the microparticle is from about 1:4 to about 1:20. In some embodiments, the ratio of height to length across the longest dimension of the microparticle is from about 1:5 to about 1:20. In some embodiments, the ratio of height to length across its longest dimension of the microparticle is from about 1:6 to about 1:20. In some embodiments, the ratio of height to length across a longest dimension of the microparticle is from about 1:8 to about 1:20. In some embodiments, the ratio of height to length across a longest dimension of the microparticle is from about 1:10 to about 1:20. In some embodiments, the ratio of height to length across a longest dimension of the microparticle is from about 1:12 to about 1:20. In some embodiments, the ratio of height to length across a longest dimension of the microparticle is from about 1:15 to about 1:20. In some embodiments, the ratio of height to length across a longest dimension of the microparticle is from about 1:4 to about 1:15. In some embodiments, the ratio of height to length across longest dimension of the microparticle is from about 1:4 to about 1:12. In some embodiments, the ratio of height to length across longest dimension of the microparticle is from about 1:4 to about 1:10. In some embodiments, the ratio of height to length at longest dimension of the microparticle is from about 1:4 to about 1:8. In some embodiments, the ratio of height to length across longest dimension of the microparticle is from about 1:4 to about 1:6.

In some embodiments, the microparticle comprises one or a plurality of asperities across its exterior surface and a height of the one or plurality of asperities comprises more than about 5% of the longest length dimension of the microparticle. In some embodiments, the microparticle comprises a height of an asperity comprising more than about 6% of the longest length. In some embodiments, the microparticle comprises a height of an asperity comprising more than about 7% of the longest length. In some embodiments, the microparticle comprises a height of an asperity comprising more than about 8% of the longest length. In some embodiments, the microparticle comprises a height of an asperity comprising more than about 9% of the longest length. In some embodiments, the microparticle comprises a height of an asperity comprising more than about 10% of the longest length.

In some embodiments, the solid substrate is in the form of a microparticle or nanoparticle and comprises about 96% silicon by weight relative to total weight of the solid substrate. In some embodiments, the solid substrate comprises about 97% silicon by weight relative to total weight of the solid substrate. In some embodiments, the solid substrate comprises about 97.5% silicon by weight relative to total weight of the solid substrate. In some embodiments, the solid substrate comprises about 98% silicon by weight relative to total weight of the solid substrate. In some embodiments, the solid substrate comprises about 98.1% silicon by weight relative to total weight of the solid substrate. In some embodiments, the solid substrate comprises about 98.2% silicon by weight relative to total weight of the solid substrate. In some embodiments, the solid substrate comprises about 98.3% silicon by weight relative to total weight of the solid substrate. In some embodiments, the solid substrate comprises about 98.4% silicon by weight relative to total weight of the solid substrate. In some embodiments, the solid substrate comprises about 98.5% silicon by weight relative to total weight of the solid substrate. In some embodiments, the solid substrate comprises about 98.6% silicon by weight relative to total weight of the solid substrate. In some embodiments, the solid substrate comprises about 98.7% silicon by weight relative to total weight of the solid substrate. In some embodiments, the solid substrate comprises about 98.9% silicon by weight relative to total weight of the solid substrate. In some embodiments, the solid substrate comprises about 99% silicon by weight relative to total weight of the solid substrate. In some embodiments, the solid substrate comprises about 99.1% silicon by weight relative to total weight of the solid substrate. In some embodiments, the solid substrate comprises about 99.2% silicon by weight relative to total weight of the solid substrate. In some embodiments, the solid substrate comprises about 99.3% silicon by weight relative to total weight of the solid substrate. In some embodiments, the solid substrate comprises about 99.4% silicon by weight relative to total weight of the solid substrate. In some embodiments, the solid substrate comprises about 99.5% silicon by weight relative to total weight of the solid substrate. In some embodiments, the solid substrate comprises about 99.6% silicon by weight relative to total weight of the solid substrate. In some embodiments, the solid substrate comprises about 99.7% silicon by weight relative to total weight of the solid substrate. In some embodiments, the solid substrate comprises about 99.8% silicon by weight relative to total weight of the solid substrate. In some embodiments, the solid substrate comprises about 99.9% silicon by weight relative to total weight of the solid substrate.

Microparticles of the disclosure can comprise any metal, including, without limitation, copper, silver, gold, platinum or palladium. Metals can also include scandium, titanium, vanadium, chromium, manganese, iron, cobalt, nickel, copper, yttrium, zirconium, niobium, molybdenum, technetium, ruthenium, rhodium, palladium, silver, hafnium, tantalum, tungsten, rhenium, osmium, iridium, platinum, gold, rutherfordium, dubnium, seaborgium, bohrium and/or hassium. In some embodiments, the microparticles or nanoparticles of the disclosure comprise a metal adhered to its exterior surface. In some embodiments, the metal is also adhered to at least one region of the interior surface of the pores or cavities. In some embodiments, the metal is positioned in the exterior surface of the microparticles with a weight that is from about 0.1% to about 7.0% of the total weight of the microparticle. In some embodiments, the metal is positioned in the exterior surface of the microparticles with a weight that is from about 0.1% to about 6.0% of the total weight of the microparticle. In some embodiments, the metal is positioned in the exterior surface of the microparticles with a weight that is from about 0.1% to about 5.0% of the total weight of the microparticle. In some embodiments, the metal is positioned in the exterior surface of the microparticles with a weight that is from about 0.1% to about 4.0% of the total weight of the microparticle. In some embodiments, the metal is positioned in the exterior surface of the microparticles with a weight that is from about 0.1% to about 3.0% of the total weight of the microparticle. In some embodiments, the metal is positioned in the exterior surface of the microparticles with a weight that is from about 0.1% to about 2.0% of the total weight of the microparticle. In some embodiments, the metal is positioned in the exterior surface of the microparticles with a weight that is from about 0.1% to about 1.0% of the total weight of the microparticle. In some embodiments, the metal is positioned in the exterior surface of the microparticles with a weight that is from about 0.1% to about 5.5% of the total weight of the microparticle. In some embodiments, the metal is positioned in the exterior surface of the microparticles with a weight that is from about 0.9% to about 7.0% of the total weight of the microparticle. In some embodiments, the metal is positioned in the exterior surface of the microparticles with a weight that is from about 0.9% to about 6.0% of the total weight of the microparticle. In some embodiments, the metal is positioned in the exterior surface of the microparticles with a weight that is from about 0.9% to about 5.0% of the total weight of the microparticle.

In some embodiments, composition and pharmaceutical compositions of the disclosure comprise microparticles or nanoparticle comprising one or a plurality of pores. In some embodiments, the pore volume is from about 0.05 to about 1.9 cm³ per gram of mass of the microparticle. In some embodiments, the pore volume is from about 0.1 to about 1.9 cm³ per gram of mass of the microparticle. In some embodiments, the pore volume is from about 0.25 to about 1.9 cm³ per gram of mass of the microparticle. In some embodiments, the pore volume is from about 0.5 to about 1.9 cm³ per gram of mass of the microparticle. In some embodiments, the pore volume is from about 0.75 to about 1.9 cm³ per gram of mass of the microparticle. In some embodiments, the pore volume is from about 1.0 to about 1.9 cm³ per gram of mass of the microparticle. In some embodiments, the pore volume is from about 1.2 to about 1.9 cm³ per gram of mass of the microparticle. In some embodiments, the pore volume is from about 1.4 to about 1.9 cm³ per gram of mass of the microparticle. In some embodiments, the pore volume is from about 1.6 to about 1.9 cm³ per gram of mass of the microparticle.

In some embodiments, the pore volume is from about 0.05 to about 1.9 cm³ per gram of mass of the microparticle. In some embodiments, the pore volume is from about 0.05 to about 1.7 cm³ per gram of mass of the microparticle. In some embodiments, the pore volume is from about 0.05 to about 1.5 cm³ per gram of mass of the microparticle. In some embodiments, the pore volume is from about 0.05 to about 1.25 cm³ per gram of mass of the microparticle. In some embodiments, the pore volume is from about 0.05 to about 1.0 cm³ per gram of mass of the microparticle. In some embodiments, the pore volume is from about 0.05 to about 0.75 cm³ per gram of mass of the microparticle. In some embodiments, the pore volume is from about 0.05 to about 0.5 cm³ per gram of mass of the microparticle. In some embodiments, the pore volume is from about 0.05 to about 0.1 cm³ per gram of mass of the microparticle.

In some embodiments, the pore volume is from about 0.1 to about 1.75 cm³ per gram of mass of the microparticle. In some embodiments, the pore volume is from about 0.25 to about 1.6 cm³ per gram of mass of the microparticle. In some embodiments, the pore volume is from about 0.5 to about 1.5 cm³ per gram of mass of the microparticle. In some embodiments, the pore volume is from about 0.75 to about 1.25 cm³ per gram of mass of the microparticle. In some embodiments, the pore volume is from about 1.0 to about 1.25 cm³ per gram of mass of the microparticle.

In some embodiment, the microparticles or nanoparticles of the disclosure comprise one or a plurality of charged ions covalently or non-covalently bound to its surface. Silicon can be functionalized with known chemical linkages or through non-directional adsorption by exposing the silicon substrate or microparticles to one or a combination of charged ionic substances. In some embodiments, the microparticles or nanoparticles of the disclosure comprise one or a combination of charged substances on its surface: arginine or other amino acids, carboxylic acids, alcohols, thiols, dyes, a molecule comprising a carboxylic acid group, or salts thereof. In some embodiments, the microparticles or nanoparticles of the disclosure comprises a net positive charge on their surfaces.

Examples of amino acids include, but are not limited to, Leucine, Tryptophan, Histidine, Valine, Lysine, Phenylalanine, Isoleucine, Threonine, Methionine, Arginine, Alanine, Glycine, Tyrosine, Proline, Cysteine, Glutamine, Asparagine, Serine, Aspartic acid and Glutamic acid.

Examples of carboxylic acids include, but are not limited to, Acetic Acid, Ascorbic Acid, Citric Acid, Acetylsalicylic Acid, Oxalic Acid, Lactic Acid, Tartaric Acid, Formic Acid, Stearic Acid, Adipic Acid, Fatty Acids, and Lauric Acid.

Examples of alcohols include, but are not limited to, ethanol, methanol, propanol, butanol, pentanol, phenol, glycerol, phenethyl alcohol, propylene glycol and isopropyl alcohol.

Examples of thiols include, but are not limited to, methanethiol, ethanethiol, coenzyme A, lipamide, glutathione, and 2-mercaptoethanol.

Examples of dyes include, but are not limited to, fluorescein, tetrachlorofluorescein, tetrabromoflurescein, fluoresceinisothiocyanate, rhodamine B, rhoadmine 6G, nile blue, and methylene blue.

The disclosure also relates to microparticle or nanoparticles that a certain BET value measured in square centimeters per gram of the microparticle or nanoparticle. In some embodiments, the microparticle or nanoparticle comprises a surface area characterized by a Brunauer, Emmett, Teller (BET) of from about 20 to about 900 centimeters squared per gram. In some embodiments, the BET value comprises a surface area of from about 20 to about 900 cm² per gram. In some embodiments, the BET value comprises a surface area of from about 30 to about 850 cm² per gram. In some embodiments, the BET value comprises a surface area of from about 40 to about 800 cm² per gram. In some embodiments, the BET value comprises a surface area of from about 50 to about 750 cm² per gram. In some embodiments, the BET value comprises a surface area of from about 60 to about 700 cm² per gram. In some embodiments, the BET value comprises a surface area of from about 70 to about 650 cm² per gram. In some embodiments, the BET value comprises a surface area of from about 80 to about 600 cm² per gram. In some embodiments, the BET value comprises a surface area of from about 90 to about 550 cm² per gram. In some embodiments, the BET value comprises a surface area of from about 100 to about 500 cm² per gram. In some embodiments, the BET value comprises a surface area of from about 125 to about 400 cm² per gram. In some embodiments, the BET value comprises a surface area of from about 150 to about 350 cm² per gram. In some embodiments, the BET value comprises a surface area of from about 200 to about 300 cm² per gram.

In some embodiments, the BET value comprises a surface area of from about 40 to about 900 cm² per gram. In some embodiments, the BET value comprises a surface area of from about 60 to about 900 cm² per gram. In some embodiments, the BET value comprises a surface area of from about 80 to about 900 cm² per gram. In some embodiments, the BET value comprises a surface area of from about 100 to about 900 cm² per gram. In some embodiments, the BET value comprises a surface area of from about 120 to about 900 cm² per gram. In some embodiments, the BET value comprises a surface area of from about 140 to about 900 cm² per gram. In some embodiments, the BET value comprises a surface area of from about 160 to about 900 cm² per gram. In some embodiments, the BET value comprises a surface area of from about 180 to about 900 cm² per gram. In some embodiments, the BET value comprises a surface area of from about 200 to about 900 cm² per gram. In some embodiments, the BET value comprises a surface area of from about 225 to about 900 cm² per gram. In some embodiments, the BET value comprises a surface area of from about 250 to about 900 cm² per gram. In some embodiments, the BET value comprises a surface area of from about 275 to about 900 cm² per gram. In some embodiments, the BET value comprises a surface area of from about 300 to about 900 cm² per gram. In some embodiments, the BET value comprises a surface area of from about 350 to about 900 cm² per gram. In some embodiments, the BET value comprises a surface area of from about 400 to about 900 cm² per gram. In some embodiments, the BET value comprises a surface area of from about 450 to about 900 cm² per gram. In some embodiments, the BET value comprises a surface area of from about 500 to about 900 cm² per gram. In some embodiments, the BET value comprises a surface area of from about 550 to about 900 cm² per gram. In some embodiments, the BET value comprises a surface area of from about 600 to about 900 cm² per gram. In some embodiments, the BET value comprises a surface area of from about 650 to about 900 cm² per gram. In some embodiments, the BET value comprises a surface area of from about 700 to about 900 cm² per gram.

In some embodiments, the BET value comprises a surface area of from about 20 to about 850 cm² per gram. In some embodiments, the BET value comprises a surface area of from about 20 to about 800 cm² per gram. In some embodiments, the BET value comprises a surface area of from about 20 to about 750 cm² per gram. In some embodiments, the BET value comprises a surface area of from about 20 to about 700 cm² per gram. In some embodiments, the BET value comprises a surface area of from about 20 to about 650 cm² per gram. In some embodiments, the BET value comprises a surface area of from about 20 to about 600 cm² per gram. In some embodiments, the BET value comprises a surface area of from about 20 to about 550 cm² per gram. In some embodiments, the BET value comprises a surface area of from about 20 to about 500 cm² per gram. In some embodiments, the BET value comprises a surface area of from about 20 to about 450 cm² per gram. In some embodiments, the BET value comprises a surface area of from about 20 to about 400 cm² per gram. In some embodiments, the BET value comprises a surface area of from about 20 to about 350 cm² per gram. In some embodiments, the BET value comprises a surface area of from about 20 to about 300 cm² per gram. In some embodiments, the BET value comprises a surface area of from about 20 to about 250 cm² per gram. In some embodiments, the BET value comprises a surface area of from about 20 to about 200 cm² per gram. In some embodiments, the BET value comprises a surface area of from about 20 to about 100 cm² per gram.

In some embodiments, the microparticles or nanoparticles of the disclosure comprise one or a plurality of active agents covalently or non-covalently bound to its surface. Silicon can be functionalized with known chemical linkages or through non-directional adsorption by exposing the silicon substrate or microparticles to one or a combination of active agents. In some embodiments, the microparticles or nanoparticles are functionalized or decorated with one or a plurality of anti-inflammatory agents, such as ibuprofen, a steroid or other non-steroidal agents.

Compositions comprising the disclosed microparticles can also comprise one or more pharmaceutically acceptable carriers, such that the composition is a pharmaceutical preparation. In some embodiments, the pharmaceutical composition of the disclosure comprises one or a plurality of microparticles or nanoparticles disclosed herein in a therapeutically effective amount. The pharmaceutical compositions of the disclosure can be in solid, liquid or gel formulations. If in a solid form, the pharmaceutical composition may be in powdered or lyophilized form.

### Methods of Making the Compositions

The compounds of the present disclosure can be made by a process for producing high specific surface area, photo luminescent porous Si (por-Si) with higher and controllable yield. In addition, the ability is demonstrated to vary the pore size distribution of mesoporous silicon including producing hierarchically structured mesoporous silicon with more than one peak in the pore size distribution.

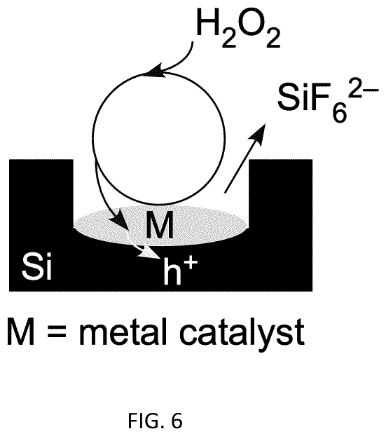

FIG. 6 depicts the disclosed injection MACE cycle for etching Si, denoted iMACE. In such a cycle, injected H₂O₂ removes an electron from a metal nanoparticle, M, which then injects a hole into the silicon substrate. The hole initiates the etching of silicon forming the etch product SiF₆²⁻, to form a pore in the substrate.

In certain embodiments, nanoparticles of any metal with a positive standard reduction potential E^{o} could be deposited galvanically, including W, Re, Bi, Cu, Po, Ru, Hg, Ag, Au, Pd, Pt, Rh, Ir, and Tl. Metals with a negative E^{o} such as Fe, Se, Pb, Sn, In, Mo, Ga, Ni, Co, Ta, Cr, and Zn would have to be deposited by some other method, e.g., evaporation or chemical vapor deposition. Metals such as W, Re, Bi, Cu, Po, and Ru that have E^{o} <0.6 will have slow kinetics for deposition as the metal onto the Si particles. These metals as well as metals with a negative E^{o} will also be susceptible to rapid dissolution in the presence of H₂O₂ in solution. The toxicities of Hg and Tl make them less attractive.

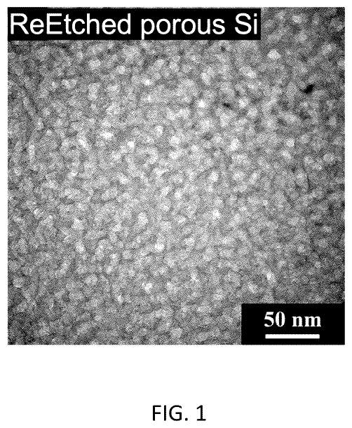

In certain embodiments, the invention uses a catalytic amount of metal deposited from an aqueous solution containing HF onto silicon powder dispersed in a solution containing acetic acid. The addition of acetic acid and the use of a pump to inject the dissolved metal ions at a controlled rate were found to allow for precise control of the amount of deposited metal as well as making the deposited layer more uniform. The deposited metal nanoparticles act as a catalyst for the reduction of an oxidant. The oxidant is injected at a controlled rate with a pump in a manner described by Kolasinski, K. W., Gimbar, N. J., Yu, H., Aindow, M., Mäkilä, E. & Salonen, J., Regenerative Electroless Etching of Silicon, Angew. Chem., Int. Ed. Engl., 56, 624-27 (2017), and in Kolasinski et al., U.S. Pat. No. 10,590,562 B2. The oxidant, as illustrated in FIG. 1(b), initiates etching by removing an electron from the metal, which then removes an electron from silicon, a process that is also called hole injection into the silicon valence band. Hole injection into the silicon valence band is a necessary condition for nanostructuring, as described in Kolasinski & Barclay, The stoichiometry of electroless silicon etching in solutions of V₂O₅ and HF, Angew. Chem., Int. Ed. Engl., 52, 6731-34 (2013). Importantly, the improved process allows the use of H₂O₂ (˜$0.5 kg⁻¹), an oxidant that is known not to produce porous Si in the absence of a metal particle catalyst, which simultaneously enhances economic viability and process control by facilitating easier thermal management, greater control over the etch rate, greater control over the extent of etching, and elimination of precipitation of impurities onto the porous powder.

More generally, disclosed is a process of electroless etching to produce porosified or hierarchically porosified semiconductor particles. The process includes the steps of: (a) providing electronics-grade, metallurgical-grade, or other semiconductor-comprising powders; (b) injecting a first oxidant, which is a metal that deposits on the surface of the semiconductor; (c) injecting a second oxidant; and (d) initiating through addition of the second oxidant nanostructure formation. This process is called injection metal assisted catalytic etching or iMACE to emphasize the importance of the injection of reagents.

### Methods of Treatment

Several embodiments of the disclosure include the use of the disclosed pharmaceutical compositions, optionally comprising pharmaceutically acceptable salt thereof on their surface, to treat dermatitis, such as atopic dermatitis, bacterial infections or symptoms associated with bacterial infection, such as edema, swelling, or fever. Such embodiment comprise administering to a subject in need thereof a pharmaceutical composition comprising a therapeutically effective amount of microparticles of the disclosure. In some embodiments, the subject is a mammal such as a human. In some embodiments, the methods comprise administering a second agent, such as an anti-infective agent or anti-inflammatory agent to the subject simultaneously, before or after administration to the subject with the disclosed pharmaceutical compositions.

Embodiments of the present disclosure are particularly useful to treat individuals who have a wound. In some embodiments, methods for treating a subject who has a wound and/or bacterial infection comprise the steps of first identifying or diagnosing the subject with an infected wound, and then administering to such a subject a therapeutically effective amount of one or a plurality of pharmaceutical compositions disclosed herein.

The disclosure also relates to a method of enhancing or accelerating wound healing in a subject in need thereof comprising exposing the pharmaceutical composition of the disclosure to a wound in the subject. In some embodiments, the step of exposing is for a time period sufficient to expose pathogenic bacteria in the wound for death of the pathogenic bacteria.

The disclosure also relates to a method of treating dermatitis in a subject in need thereof comprising administering to the subject a therapeutically effective amount of the pharmaceutical composition of the disclosure. The disclosure also relates to a method of treating atopic dermatitis in a subject in need thereof comprising administering to the subject a therapeutically effective amount of the pharmaceutical composition of the disclosure.

In some embodiments of the present disclosure, methods for treating a subject who has been identified as having a wound and/or bacterial infection comprise administering to such an individual a therapeutically effective amount of the microparticle of the disclosure which is known to be effective to inhibit cell migration in greater than 50% of cells in an in vitro migration assay at a concentration of less than 1 mM.

In some embodiments, any of the methods disclosed herein are free of a step of administering to the subject a second agent other than the disclosed pharmaceutical composition.

Any pathogenic bacterial strain within a subject may be treated by way of administration of the composition to a subject in need of treatment. Strains of bacteria that may be treated include:
Staph
Pathogenic E. coli; and
Methicillin-resistant forms of the aforementioned species.

### Pharmaceutical Compositions and Routes of Administration

In some embodiments, the present disclosure provides compositions comprising any one or more of the active agents described herein, either alone or in combination, for example for use in treating a wound and/or bacterial infection. For example, in some embodiments, the present disclosure provides compositions comprising a microparticle disclosed herein, or a solid substrate disclosed herein, for example, for use in treating wounds and/or bacterial infections.

Pharmaceutical compositions provided by the present disclosure include compositions wherein the active microparticle or nanoparticle (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., microparticle), and/or reducing, eliminating, or slowing the progression of disease symptoms (e.g. symptoms of a wound and/or bacterial infection). Determination of a therapeutically effective amount of a compound of the disclosure is well within the capabilities of those skilled in the art, especially in light of the detailed disclosure herein.

The pharmaceutical composition may be formulated by one having ordinary skill in the art with compositions selected depending upon the chosen mode of administration. Suitable pharmaceutical carriers are described in the most recent edition of Remington's Pharmaceutical Sciences, A. 0501, a standard reference text in this field, which is incorporated herein in its entirety.

Administering the pharmaceutical composition can be effected or performed using any of the various methods known to those skilled in the art. Systemic formulations include those designed for administration by injection, e.g. subcutaneous, intravenous, intramuscular, intrathecal or intraperitoneal injection, as well as those designed for transdermal, transmucosal, oral or pulmonary administration. In some embodiments, administration of the effective amount of pharmaceutical composition disclosed herein is not limited to any particular delivery system and includes, without limitation, parenteral (including subcutaneous, intravenous (via injection or infusion), intramedullary, intraarticular, intramuscular, or intraperitoneal injection), rectal, topical, transdermal, mucosal or oral (for example, in capsules, suspensions, or tablets) administration. In some embodiments, administration to a subject in need thereof occurs in a single dose or in repeat administrations, and in any of a variety of physiologically acceptable salt forms, or with an acceptable pharmaceutical carrier or additive as part of a pharmaceutical composition. In some embodiments, any suitable and physiological acceptable salt forms or standard pharmaceutical formulation techniques, dosages, and excipients are utilized. In some embodiments, the step of administering comprises administering the composition or pharmaceutical composition intravenously, intramuscularly, topically, intradermally, intramucosally, subcutaneously, sublingually, orally, intravaginally, intracavernously, intraocularly, intranasally, intrarectally, gastrointestinally, intraductally, intrathecally, subdurally, extradurally, intraventricular, intrapulmonary, into an abscess, intraarticularly, into a bursa, subpericardially, into an axilla, intrauterine, into the pleural space, intraperitoneally, transmucosally, or transdermally.

Pharmaceutical compositions of the invention may be administered by a variety of routes including oral, buccal, sublingual, rectal, transdermal, subcutaneous, intravenous injection, intravenous infusion, intramuscular, intrathecal, intraperitoneal and intranasal. Depending on whether intended route of delivery is oral or parenteral, the active agents can be formulated as compositions that are, for example, either injectable, topical or oral compositions. Liquid forms of compositions may include a suitable aqueous or nonaqueous vehicle with buffers, suspending and dispensing agents, colorants, flavors and other suitable ingredients known in the art. Solid forms of compositions may include, for example, binders, excipients, lubricants, coloring agents, flavoring agents and other suitable ingredients known in the art. The active agents and pharmaceutical compositions described herein may also be administered in sustained release forms or from sustained release drug delivery systems known in the art.

The pharmaceutical composition may depend on the disease or condition and on whether the administration is to prevent or to treat the disease or condition. For instance, administration for prevention of several diseases or conditions, including, but not limited to bacterial infection, atopic dermatitis or wound healing.

In some embodiments the compositions of the present disclosure are pharmaceutical compositions comprising one or more active agents, one or more microparticles, together with one or more conventionally employed components suitable for use in pharmaceutical delivery such as carriers, stabilizers, diluents, dispersing agents, suspending agents, thickening agents, excipients, and the like, may be placed into the form of pharmaceutical formulations. Nonlimiting examples of such formulations include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration. The pharmaceutical compositions and formulations described herein may, where appropriate, be conveniently presented in discrete unit dosage forms and may be prepared by any of the methods well known in the art of pharmacy. Such methods include the step of bringing into association an active agent with liquid carriers, solid matrices, semi-solid carriers, finely divided solid carriers or combinations thereof, and then, if necessary, shaping the product into the desired delivery system. Unit dosage forms of a pharmaceutical composition or formulation preferably contain a predetermined quantity of active agent and other ingredients calculated to produce a desired therapeutic effect, such as an effective amount of a therapeutically effective amount. Typical unit dosage forms include, for example, prefilled, premeasured ampules or syringes of liquid compositions, or pills, tablets, capsules or the like for solid compositions.

For parenteral administration, the microparticle can be, for example, formulated as a solution, suspension, emulsion or lyophilized powder in association with a pharmaceutically acceptable parenteral vehicle or pharmaceutically acceptable carrier. Examples of such vehicles or carriers are water, saline, Ringer's solution, dextrose solution, 5% human serum albumin, Ringers dextrose, dextrose and sodium chloride, lactated Ringers and fixed oils, polyethylene glycol, polyvinyl pyrrolidone, lecithin (glycerophospholipids), arachis oil or sesame oil. Liposomes and nonaqueous vehicles such as fixed oils may also be used. The vehicle or lyophilized powder may contain additives that maintain isotonicity (e.g, sodium chloride, mannitol) and chemical stability (e.g., buffers and preservatives). The formulation is sterilized by commonly used techniques. Parenteral dosage forms may be prepared using water or another sterile carrier. For example, a parenteral composition suitable for administration by injection is prepared by dissolving 1.5% by weight of active ingredient in 0.9% sodium chloride solution. Alternatively, the solution can be lyophilized and then reconstituted with a suitable solvent just prior to administration.

Pharmaceutically acceptable carriers are well known to those skilled in the art and include, but are not limited to, from about 0.01 to about 0.1 M or about 0.05 M phosphate buffer or about 0.8% saline. Intravenous carriers include fluid and nutrient replenishers, electrolyte replenishers such as those based on Ringer's dextrose, and the like. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media.

The pharmaceutical compositions can be prepared using conventional pharmaceutical excipients and compounding techniques. Oral dosage forms may be elixirs, syrups, tablets, pills, dragees, capsules, liquids, gels, syrups, slurries, suspensions and the like, for oral ingestion by a patient to be treated. The typical solid carrier may be an inert substance such as lactose, starch, glucose, cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; binding agents, magnesium stearate, dicalcium phosphate, mannitol and the like. A composition in the form of a capsule can be prepared using routine encapsulation procedures. For example, pellets containing the active ingredient can be prepared using standard carrier and then filled into a hard gelatin capsule; alternatively, a dispersion or suspension can be prepared using any suitable pharmaceutical carrier(s), for example, aqueous gums, celluloses, silicates or oils and the dispersion or suspension then filled into a soft gelatin capsule. Typical liquid oral excipients include ethanol, glycerol, glycerin, non-aqueous solvent, for example, polyethylene glycol, oils, or water with a suspending agent, preservative, flavoring or coloring agent and the like. All excipients may be mixed as needed with disintegrants, diluents, lubricants, and the like using conventional techniques known to those skilled in the art of preparing dosage forms. If desired, disintegrating agents may be added, such as the crosslinked polyvinylpyrrolidone, agar, or alginic acid or a salt thereof such as sodium alginate. If desired, solid dosage forms may be sugarcoated or coated using standard techniques, including, but not limited to the use of chitosan, to target specific regions of the gastrointestinal tract. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compositions of the disclosure may take the form of tablets, lozenges, and the like formulated in conventional manner. The compounds may also be formulated in rectal or vaginal compositions such as suppositories or enemas. A typical suppository formulation comprises a binding and/or lubricating agent such as polymeric glycols, glycerides, gelatins or cocoa butter or other low melting vegetable or synthetic waxes or fats. For administration by inhalation, the compounds for use according to the present disclosure are conveniently delivered in the form of an aerosol spray from pressurized packs or a nebulizer, with the use of a suitable propellant, e.g., di- or trichlorofluoromethane, dichlorotetrafluoroethane, carbon dioxide or other suitable gases. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The formulations may also be a depot preparation which can be administered by implantation (for example subcutaneously or intramuscularly) or by intramuscular injection. In such embodiments, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used. Certain organic solvents such as dimethylsulfoxide also may be employed, although usually at the cost of greater toxicity. Additionally, the compounds may be delivered using a sustained release system, such as semipermeable matrices of solid polymers containing the therapeutic agent. Various of sustained-release materials have been established and are well known by those skilled in the art. Sustained release capsules may, depending on their chemical nature, release the compounds for a few weeks up to over 100 days. Depending on the chemical nature and the biological stability of the therapeutic reagent, additional strategies for protein stabilization may be employed.

The compounds described herein may also be formulated for parenteral administration by bolus injection or continuous infusion and may be presented in unit dose form, for instance as ampoules, vials, small volume infusions or prefilled syringes, or in multi-dose containers with an added preservative.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

The dose of microparticles described herein may be calculated based on studies in humans or other mammals carried out to determine efficacy and/or effective amounts of the active agent (see section E, Clinical Outcomes, below). The dose amount and frequency or timing of administration may be determined by methods known in the art and may depend on factors such as pharmaceutical form of the active agent and route of administration, and patient characteristics including age, body weight or the presence of any medical conditions affecting drug metabolism.

In one embodiment, a dose may be administered as a single dose. In another embodiment, a dose may be administered as multiple doses over a period of time, for example, at specified intervals, such as, daily, bi-weekly, weekly, monthly, and the like. In another embodiment, the dose will be 700 mg/kg/d (Table 3, 2010-04-15). In another embodiment, the dose will be increased over time until early signs of renal or cytotoxicity are observed, in which case the dose level will be decreased to the previous, well-tolerated level.

In some embodiments, the dose of active agent is about at least about 10 mg, at least about 20 mg, at least about 30 mg, at least about 40 mg, at least about 50 mg, at least about 75 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1200 mg, at least about 1500 mg, at least about 2000 mg, at least about 2500 mg, at least about 3000 mg, at least about 4000 mg, at least about 5000 mg, at least about 7500 mg, at least about 10,000 mg, at least about 15,000 mg, at least about 20,000 mg, or at least about 25,000 mg. In some embodiments, the above dosages are mg/day or mg/kg/day. In another embodiment, the dose of active agent is in the range of from about 1 to about 10000 mg, from about 1 to about 7500 mg, from about 1 to about 5000 mg, from about 1 to about 2500 mg, from about 1 to about 1000 mg, from about 1 to about 500 mg, from about 1 to about 250 mg, from about 250 to about 10000 mg, from about 250 to about 5000 mg, from about 250 to about 1000 mg, from about 250 to about 500 mg, from about 500 to about 10000 mg, from about 500 to about 5000 mg, from about 500 to about 1000 mg. In some embodiments, the above dosages are mg/day or mg/kg/day. In some embodiments, the dose of the disclosed pharmaceutical compositions is from about 1 to about 20 grams per day. In some embodiments, the dose of pharmaceutical compositions is from about 10 to about 20 grams per day.

In some embodiments, a single dose may be administered. In another embodiment, multiple doses may be administered over a period of time, for example, at specified intervals, such as, four times per day, three times a day, twice per day, once a day, weekly, monthly, 4 times over 14 days, 2 times over 21 days, twice per month, 4 times over 21 days, 4 times per month, or 5, 6, 7, 8, 9, 10, 11, 12 or more times per month, per 21 days, per 14 days, or per week, and the like.

The methods and compositions described herein may be used to treat or prevent wounds and/or bacterial infections, in any subject in need of such treatment. In one embodiment, the subject is a human, horse, cow, pig, sheep, goat, dog, cat, or other domesticated animal. In some embodiments, the subjects can be of any age, ranging from newborns to older adults. In some embodiments, it may be desirable to treat young subjects, for example young infants. Similarly, in some embodiments it may be desirable to treat much older subjects, particularly where such subjects begin to exhibit indicators or symptoms of deficiencies in wound healing or bacterial immunity.

The methods and compositions described herein may be employed as prophylactic treatments or therapeutic treatments. For prophylactic treatments, the methods and compositions provided herein can be used preventatively in subjects that do not yet exhibit any clear or detectable clinical indicators or symptoms of bacterial infection. A subject receiving prophylactic treatment for bacterial infection, for example, may not exhibit any clinical indicators or symptoms of bacterial infection. In the case of therapeutic treatments, the methods and compositions provided herein can be used in subjects that already exhibit one or more clinical indicators or symptoms of the disease or disorder, such as atopic dermatitis. A subject receiving therapeutic treatment for atopic dermatitis, for example, may have been clinically diagnosed with dermatitis.

In one embodiment, a subject may have been identified as being at risk of developing a wound and/or bacterial infection. In one embodiment, the subject has one or more genetic risk factors associated with developing a wound and/or bacterial infection. In one embodiment, a subject may have been identified as being at risk of developing atopic dermatitis.

In some embodiments the methods of treatment provided herein (which comprise, for example, administering to a subject an effective amount of a composition according to the present disclosure) result in, or are aimed to achieve, a detectable improvement in one or more clinical indicators or symptoms of atopic dermatitis.

In the context of bacterial infections, for instance, the clinical indicators or symptoms include, but not limited to, redness, edema, swelling and fever. In some embodiment of the present disclosure a symptom or indicator of improvement is selected from the group comprising survival, disease-free survival, results of a blood test and/or reduction in edema, reduction in swelling, reduction of fever or accelerated healing at the site of a wound on the skin of a subject.

### Additional Methods

The disclosure relates to methods of breaking up the mucilage mass comprising exposing mucilage mass to a composition comprising any of the disclosed microparticles or nanoparticles.

The disclosure also relates to a method of decontaminating a surface or fluid contaminated with pathogenic bacteria by exposing the contaminated surface or fluid to a composition comprising any of the disclosed microparticles or nanoparticles. In some embodiments, the surface may be the surface of food. In other embodiments, the contaminated fluid is exposed to the composition in a filter or other device that holds a solid or powdered form of the disclosed compositions.

The compositions and methods described herein are illustrative only and not intended to be limiting. Those of skill in the art will appreciate that various combinations or modifications of the specific compositions and methods described above can be made, and all such combinations and modifications of the compositions and methods described herein may be used in carrying out the present disclosure. Furthermore, certain embodiments of the present disclosure are further described in the following non-limiting Examples, and also in the following Claims.

### REFERENCES

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.
Ashtikar, M. & Wacker, M. G. (2018) Nanopharmaceuticals for wound healing—Lost in translation? Adv. Drug Deliver. Rev., 129, 194-218.
Burrell, R. E., Heggers, J. P., Davis, G. J. & Wright, J. B. (1999) Efficacy of silver-coated dressings as bacterial barriers in a rodent burn sepsis model. Wounds—a Compendium of Clinical Research and Practice, 11, 64-71.
Chen, X., Wo, F., Jin, Y., Tan, J., Lai, Y. & Wu, J. (2017) Drug-Porous Silicon Dual Luminescent System for Monitoring and Inhibition of Wound Infection. ACS Nano, 11, 7938-7949.
Dalilottojari, A., Delalat, B., Harding, F. J., Cockshell, M. P., Bonder, C. S. & Voelcker, N. H. (2016) Porous Silicon-Based Cell Microarrays: Optimizing Human Endothelial Cell-Material Surface Interactions and Bioactive Release. Biomacromolecules, 17, 3724-3731.
Dashnyam, K., Jin, G.-Z., Kim, J.-H., Perez, R., Jang, J.-H. & Kim, H.-W. (2017) Promoting angiogenesis with mesoporous microcarriers through a synergistic action of delivered silicon ion and VEGF. Biomaterials, 116, 145-157.
Dipietro, J. A. & Kolasinski, K. W. (2022) Characterization of Mechanochemical Modification of Porous Silicon with Arginine. Surfaces, 5, 143-154.
Föll, H., Christophersen, M., Carstensen, J. & Hasse, G. (2002) Formation and application of porous silicon. Mater. Sci. Eng., R, 39, 93-141.
Hasanzadeh Kafshgari, M., Delalat, B., Harding, F. J., Cavallaro, A., Mäkilä, E., Salonen, J., Vasilev, K. & Voelcker, N. H. (2016) Antibacterial properties of nitric oxide-releasing porous silicon nanoparticles. J. Mater. Chem. B, 4, 2051-2058.
Jahromi, M. a. M., Zangabad, P. S., Basri, S. M. M., Zangabad, K. S., Ghamarypour, A., Aref, A. R., Karimi, M. & Hamblin, M. R. (2018) Nanomedicine and advanced technologies for burns: Preventing infection and facilitating wound healing. Adv. Drug Deliver. Rev., 123, 33-64.
Kafshgari, M. H., Cavallaro, A., Delalat, B., Harding, F. J., Mcinnes, S. J., Mäkilä, E., Salonen, J., Vasilev, K. & Voelcker, N. H. (2014) Nitric oxide-releasing porous silicon nanoparticles. Nanoscale Res. Lett., 9, 333.
Mäkila, E., Anton Willmore, A.-M., Yu, H., Irri, M., Aindow, M., Teesalu, T., Canham, L. T., Kolasinski, K. W. & Salonen, J. (2019) Hierarchical Nanostructuring of Porous Silicon with Electrochemical and Regenerative Electroless Etching. ACS Nano, 13, 13056-13064.
Neidrauer, M., Ercan, U. K., Bhattacharyya, A., Samuels, J., Sedlak, J., Trikha, R., Barbee, K. A., Weingarten, M. S. & Joshi, S. G. (2014) Antimicrobial efficacy and wound-healing property of a topical ointment containing nitric-oxide-loaded zeolites. J. Med. Microbiol., 63, 203-209.
Quirynen, M. & Bollen, C. M. L. (1995) The influence of surface roughness and surface-free energy on supra- and subgingival plaque formation in man. Journal of Clinical Periodontology, 22, 1-14.
Sadrearhami, Z., Nguyen, T.-K., Namivandi-Zangeneh, R., Jung, K., Wong, E. H. H. & Boyer, C. (2018) Recent advances in nitric oxide delivery for antimicrobial applications using polymer-based systems. J. Mater. Chem. B, 6, 2945-2959.
Scarano, A., Piattelli, M., Caputi, S., Favero, G. A. & Piattelli, A. (2004) Bacterial Adhesion on Commercially Pure Titanium and Zirconium Oxide Disks: An In Vivo Human Study. Journal of Periodontology, 75, 292-296.
Tamarov, K., Kiviluoto, R., Swanson, J. D., Unger, B. A., Ernst, A. T., Aindow, M., Riikonen, J., Lehto, V.-P. & Kolasinski, K. W. (2020a) Low-Load Metal-Assisted Catalytic Etching Produces Scalable Porosity in Si Powders. ACS Appl. Mater. Interfaces, 12, 48969-48981.
Tamarov, K., Swanson, J. D., Unger, B. A., Kolasinski, K. W., Ernst, A. T., Aindow, M., Lehto, V.-P. & Riikonen, J. (2020b) Controlling the nature of etched Si nanostructures: High versus low load metal-assisted catalytic etching (MACE) of Si powders. ACS Appl. Mater. Interfaces, 12, 4787-4796.
Teughels, W., Van Assche, N., Sliepen, I. & Quirynen, M. (2006) Effect of material characteristics and/or surface topography on biofilm development. Clinical Oral Implants Research, 17, 68-81.
Zawislak, J. P., Kolasinski, K. W. & Helmke, B. P. (2009) Development of endothelial cells on pillar-covered silicon. Phys. Status Solidi A, 206, 1356-1360.
Zeng, Q. Y., Han, K., Zheng, C. Y., Bai, Q., Wu, W. D., Zhu, C. H., Zhang, Y. N., Cui, N. & Lu, T. L. (2022) Degradable and self-luminescence porous silicon particles as tissue adhesive for wound closure, monitoring and accelerating wound healing. J. Colloid Interface Sci., 607, 1239-1252.

### EXAMPLES

### Example 1

The antibacterial action of rough por-Si particles and rough por-Si doped with Cu nanoparticles have been investigated. Porous silicon microparticles were produced using an LL-MACE process as described in U.S. 62/881,636, and incorporated by reference in its entirety, to porosity highly p-type doped Si powder with a particle size ≤45 μm. A low load of Cu was used as the catalyst for the LL-MACE process. Additional CuNP are deposited onto the external surfaces of the porosified particles by addition of Cu²⁺(aq) by deposition methods such as those described in U.S. 62/881,636. SiNP that contain no CuNP were produced by ReEtching of metallurgical grade Si powder with a particle size ≤45 μm with the procedures described in U.S. Pat. No. 10,590,562 B2, which is incorporated by reference in its entirety. SiNP were produced by grinding of por-Si microparticles produce as described by either process. The SiNP had a mean hydrodynamic diameter of 300 nm as determined by dynamic light scattering (DLS).

Culturing of E. coli and S. aureus was performed in an agar gel. Compared to a control with no added nanoparticles (defined as 100% after 1 h) the colony growth rates were reduced to 65% and 85% for E. coli and S. aureus, respectively, when 0.1 mg/ml of SiNP were added. When 0.7 wt % Cu is added to the SiNP, which were again added to the gel at a concentration of 0.1 mg/ml, no measurable bacterial growth was observed, thus the growth rate was 0%. Hence, rough SiNP produced by grinding ReEtched por-Si exhibit antibacterial activity that can be enhanced by Cu-doped SiNP exhibit enhanced antibacterial activity that is superior to both.

### Example 2

The differential functionalization of exterior and interior surface of por-Si particles was investigated. As described by DiPietro and Kolasinski (DiPietro and Kolasinski, 2022), por-Si particles were produced by ReEtching of metallurgical grade Si powder with a particle size≤ 45 μm with the procedures described in U.S. Pat. No. 10,590,562 B2. Jar milling of a mixture of por-Si and arginine was performed using borundum grinding media for various lengths of time extending to 4 h. Infrared absorption spectroscopy was used to confirm attachment of the arginine to the newly formed exterior surface. Rinsing to remove unreacted arginine as well as any physisorbed products reveals that the covalent attachment has been achieved. Covalent attachment to the external surface through the formation of an Si—O linkage with the NH₂ moiety of the arginine reactant pointed away from the surface was confirmed not only by the infrared absorption spectrum but also by the change from hydrophobic (for the as-etched hydrogen-terminated particles) to hydrophilic (for the arginine-terminated particles) behavior. The hydrophilic arginine-terminated particles are easily dispersed in water and exhibit a positive zeta potential, again consistent with NH₂ groups extending away from the particle exterior surface. Simultaneously, the observation of residual Si—H infrared absorption indicates hydrogen atoms remain attached to the internal surface of the remaining pores. Fracturing of por-Si during grinding generates smaller particles with freshly created Si dangling bonds not terminated with H atoms. These dangling bonds drive the reaction that attaches the arginine to the surface. Since interior surface remained H-terminated, only the exterior surfaces reacted with arginine. This indicates that particles with reduced size were created with different functionalizations on their interior and exterior surfaces. In other words, the outside of the particles was covered with covalently bound arginine while the internal pore walls were covered primarily with H atoms.

### Example 3

The loading of model small molecule drugs into por-Si powders was investigated. Porous silicon microparticles were produced using an LL-MACE process as described in U.S. 62/881,636 to porosify highly p-type doped Si powder with a particle size≤ 45 μm. A low load of Cu was used as the catalyst for the LL-MACE process to produce particles with a mean pore size in the range of 8-10 nm. The particles were oxidized for 4 h at 300° C. in air. This creates a thin suboxide surface layer SiOₓ in which x<2 terminated with OH groups. This process transforms the hydrophobic as-etched particles into hydrophilic particles. Ibuprofen or lidocaine was loaded into the pores using conventional methods such as infusion from alcoholic solutions or incipient wetting from a melt of the pure drug compound. Loading was confirmed by differential scanning calorimetry (DSC). In the case of ibuprofen, the kinetics of release were followed using UV/Vis absorption spectroscopy. Ibuprofen exhibits an absorption maximum at 264 nm. Release of ibuprofen into a phosphate buffered saline solution (PBS) was confirmed by UV/Vis spectroscopy. The PBS was extracted, spectroscopically analyzed, and replaced periodically to follow the concentration of ibuprofen released over the course of several days. Initial burst release was followed by a decaying concentration profile that is approximately exponential in nature, consistent with diffusion of ibuprofen out of the pores.

Both ibuprofen and lidocaine are polar molecules. Loading of these polar drugs into por-Si with hydrophobic H-terminated surfaces was significantly reduced. Loading of these polar drugs into por-Si with hydrophilic OH-terminated surfaces was greatly enhanced. This demonstrates the importance of optimized surface chemistry in the loading process. Sufficiently strong drug-surface interactions are required to facilitate drug loading. Loading of drugs into the pores protects the drugs from degradation in solution. The protection arises both because of exclusion of the solution from the pores that are filled with the drug but also by attraction of the drug molecules to the pore walls, which sterically hinders the reaction of the drug with any other molecule. By judicious choice of surface chemistry by, e.g., oxidation, hydrosilylation, mechanochemistry, photochemistry, laser ablation, etc., the drug-wall interactions can be modulated to enhance protection and the rate of release from the pores. This can be beneficial both to enhance shelf life and to facilitate extended time release of the drug.

S. Aureus Growth Inhibition after 1 hr. Incubation

1 mg of ZnO or Cu-doped porous Si per ml of agar gel is mixed into the gel prior to introduction of the bacterial culture. The number of bacteria is quantified in colony-forming units CFU per milliliter. ZnO nanoparticles (<100 nm) are used as a control compared to 20 μm Cu-doped porous Si particles. The extent of Cu doping is expressed by the ratio of the mass of Cu to the mass of porous Si (Cu/Si). See Table 1.

TABLE 1

| | Cu/Si | N (CFU/ml) | Nₓ/N_{control} | Log(Nₓ/N_{control}) |
|---|---|---|---|---|
| No NP control | | 133000000 | 1 | 0 |
| ZnO | | 650000 | 4.89E−03 | −2.31 |
| A | 0.00794 | 200 | 1.50E−06 | −5.82 |
| B | 0.0159 | 150 | 1.13E−06 | −5.948 |
| C | 0.0318 | 80 | 6.02E−07 | −6.22 |
| D | 0.0635 | 840 | 6.32E−06 | −5.20 |
| E | 0.1589 | 20 | 1.50E−07 | −6.82 |

### Antibacterial Additions to Footbaths for Cattle

The Merck Veterinary Manual (www.merckvetmanual.com/musculoskeletal-system/lameness-in-cattle/footbaths-of-cattle) recommends a 5% solution of copper sulfate for control of interdigital dermatitis and footrot (interdigital phlegmon). This has an advantage over 4% formalin because formalin is a hazardous waste. Nonetheless, spent copper sulfate solutions need to be diluted to the highest practical extent before spreading widely on land to avoid overaccumulation in soil. Copper-doped porous silicon exhibits antibacterial activity using over 30,000 times less copper than treatment with copper sulfate. Therefore, the risk of overaccumulation of copper is greatly minimized. In addition, porous silicon added to a manure slurry degrades into a bioavailable form of silicon, which is designated as a plant beneficial substance by the Association of American Plant Food Control Officials (AAPFCO). According to the New Jersey Agricultural Experiments Station (njaes.rutgers.edu/fs1278/), the benefits of silicon nutrition include stimulation of growth and yield, counteracting negative effects of excess N nutrition, suppression of bacterial and fungal infections, suppression of some insect infestations, and improved tolerance to environmental stress. Copper-doped porous silicon is beneficial both because of its high antibacterial efficacy as well as its favorable disposal characteristics.

Hoof treatment used in this study is an antimicrobial formula that does not contain antibiotics. It is a colloidal suspension of porous silicon in a hyperosmotic citrate buffer. The porous silicon particles are themselves decorated with copper nanoparticles to enhance their antibacterial and wound healing properties.

Hoof Treatment was applied by spraying the affected area after the affected area has first been cleaned with water to remove dirt and debris. Shake before using. A bandage may be applied to the affected area. The pH 6 Citrate buffer facilitates acidic irrigation of alkaline wounds at close to normal skin pH. The Hoof treatment was a hyperosmotic, hypertonic solution that helps clear wound of infectious matter and promotes wound healing.

The formulation tried was 0.1 wt % dispersion of Cu-coated porous Si microparticles in a citrate buffer. The pharmaceutical composition comprises water, sodium citrate, citric acid, silicon, copper and purple food color. Recommended spray dose was about 5 to about 10 ml of liquid solution. Apply after hooves have been cleaned with water and allow 15-20 min to dry (e.g. during milking) before releasing back to pen/pasture.

### Sea Snot

Cells have been shown to preferentially attach to and proliferate on rough rather than smooth surfaces. Therefore, the rough nanostructured surfaces of porous silicon may be effective at incorporating into marine mucilage, suppressing phytoplankton and bacterial growth, and breaking up the mucilage mass. The porous silicon itself is nontoxic to most marine life as it decomposes into silica and bioavailable silicic acid. Porous silicon powder applied directly on top of marine mucilage under solar irradiation may prove to be an effective remedy against marine mucilage. A potential method of boasting the effectiveness of porous silicon powder is to incorporate copper nanoparticles into the porous silicon. Incorporating copper nanoparticles into porous silicon has the advantage of delivering the strong antimicrobial properties of copper, while reducing the copper burden on the environment. The rough surfaces of porous silicon enhance uptake of the silicon particles by microbes, which are then exposed to copper ions released from the copper-doped porous silicon particles. Upon microbe death, the porous silicon particles (with or without copper doping) are released to be incorporated into more microbes until they are finally dissolved away and used in remediation applications, for example, as an amendment to biosand filters not only as an antimicrobial agent but also to remove certain metals.

What is claimed is:

1. A composition comprising a solid substrate comprising silicon greater than about 90% in weight relative to total weight of the solid substrate and greater than about 0.1% weight in oxygen relative to the total weight of the solid substrate;
wherein the solid substrate is in the form of a microparticle comprising an exterior surface and one or a plurality of pores defining one or a plurality of interior surfaces;
wherein the solid substrate is no more than about 500 microns at its greatest length; and wherein at least about 50% of the exterior surface comprises protrusions or asperities and/or a relative roughness of about 1% to about 10% of its exterior surface.

2. The composition of claim 1, wherein the exterior surface comprises a metal.

3. The composition of claim 2, wherein the metal is adhered onto the exterior surface in the form of nanoparticles.

4. The composition of claim 2, wherein the metal is copper, silver, gold, platinum or palladium.

5. The composition of claim 1, wherein the solid substrate comprises silicon at about 98% in weight relative to total weight of the solid substrate.

6. The composition of claim 1, wherein the solid substrate comprises silicon at about 99% in weight relative to total weight of the solid substrate and wherein the exterior surface comprises nanoparticles of metal.

7. The composition of claim 1, wherein the exterior surface comprises a region of hydrophilicity.

8. The composition of claim 7, wherein the solid substrate in an aqueous solution has a zeta potential from about +20 to about +80 mV.

9. The composition of claim 7, wherein the region of hydrophilicity across the exterior surface comprises polar adsorbates.

10. (canceled)

11. The composition of claim 1, wherein the solid substrate is in powdered form.

12. (canceled)

13. The composition of claim 1, wherein the microparticle Brunauer, Emmett, Teller (BET) comprises a surface area of from about 20 to about 900 centimeters squared per gram.

14. The composition of claim 1, wherein the microparticle: (a) is pseudo-rectangular in shape and comprises a longest dimension in length from about 1 to about 75 microns, (b) has a longest dimension across of about 7.5 microns; (c) comprises a height of an asperity comprising more than about 5% of the longest length; (d) comprises copper and a weight to weight ratio copper to silicon of from about 0.1 to about 7.0%; or (e) comprises a pore volume of from about 0.05 to about 1.9 cm³ per gram of mass of the microparticle.

15. (canceled)

16. The composition of claim 1, wherein the ratio of height to length at longest dimension of the microparticle is from about 1:4 to about 1:20.

17-18. (canceled)

19. The composition of claim 1, wherein the exterior surface of the microparticle is bound to a therapeutic agent.

20. A pharmaceutical composition comprising: (i) the composition of claim 1; and (ii) a pharmaceutically acceptable carrier.

21. The pharmaceutical composition of claim 20, wherein the composition is in a solid dosage form or a liquid dosage form; and wherein the solid substrate is at a weight from about 0.1% to about 5.0% weight to weight ratio of the solid substrate to total weight of the pharmaceutical composition.

22. A method of treating a bacterial infection in a subject in need thereof comprising administering to the subject a therapeutically effective amount of the pharmaceutical composition of claim 20.

23. (canceled)

24. A method of enhancing or accelerating wound healing in a subject in need thereof comprising exposing the pharmaceutical composition of claim 20 to a wound in the subject.

25. A method of treating dermatitis in a subject in need thereof comprising administering to the subject a therapeutically effective amount of the pharmaceutical composition of claim 20.

26. A method of making the composition of claim 1 comprising etching a composition of silicon with an initial amount of a first oxidant reactive with silicon.

27-33. (canceled)
